(19) United States
(12) Patent Application Publication
(10) Pub. No.: US 2004/0167217 A1
(43) Pub. Date: Aug. 26, 2004

(54) Neuroprotective effects of polyphenolic compounds

(76) Inventors: Giovanni Scapagnini, Gaithersburg, MD (US); Daniel L. Alkon, Bethesda, MD (US); Vittorio Calabrese, Acicastello (IT); Roberto Motterlini, Watford (GB); Claudia Colombrita, Catania (IT)

Correspondence Address: MILBANK, TWEED, HADLEY & MCCLOY LLP, INTERNATIONAL SQUARE BUILDING, 1825 EYE STRET, N.W. #1100, WASHINGTON, DC, 20006, US

(21) Appl. No.: 10/420,465

(22) Filed: Apr. 22, 2003

Related U.S. Application Data
(60) Provisional application No. 60/450,044, filed on Feb. 26, 2003.

(51) Int. Cl. A61K 31/35; A61K 31/235; A61K 31/12

(52) U.S. Cl. 514/543; 514/679

(57) ABSTRACT

The invention relates to a class of compounds, and analogs thereof, which are effective in protecting cells of the central and peripheral nervous system from deterioration and cell death arising from degenerative disease, trauma, aging or like condition, disease or disorder. The methods utilize novel anti-apoptotic and neuroprotective effects for a group of natural polyphenols in cells of the central and peripheral nervous system.

### FIELD OF THE INVENTION

The invention relates to the general field of medicinal chemistry and treatments and prevention for diseases, conditions and disorders of the nervous system. In particular, the invention relates to a class of compounds, and analogs thereof, which are effective in protecting cells of the central and peripheral nervous system from deterioration and cell death arising from degenerative disease, trauma, aging, and the like.

### BACKGROUND OF THE INVENTION

Neurodegenerative diseases or disorders are characterized by progressive neuronal cell death. Amyotrophic lateral sclerosis (ALS), Alzheimer's disease, and Parkinson's disease are examples of neurodegenerative disorders characterized by progressive neuronal cell death. The effects of neuronal cell death are especially alarming since these cells do not readily regenerate. However, neuroprotection is a beneficial effect that may result in salvage, recovery or regeneration of the nervous system, its cells, structure and function. Therefore, targeting neuroprotection and cell death or apoptosis using certain drugs, small molecules, or compounds may be beneficial in treating cell death associated diseases, disorders, or conditions, such as neurodegenerative diseases or disorders.

Apoptosis has long been implicated in neurodegeneration. Apoptosis is a morphologically and biochemically defined form of cell death caused by active cellular signaling. Morphologically, apoptosis of a cell occurs as follows: condensation of the nucleus of the cell; cell shrinkage; cytoplasmic vacuolation and cell surface smoothing; enlargement of intercellular space; release of the cell from the pericellular region; fragmentation of the cell (to provide apoptosis body) and phagocytosis of the fragment by macrophages or the like. Biochemically, nucleosomal DNA is cleaved by endonuclease into 180-220 base pairs DNA fragments¹. It has been revealed that apoptosis plays a role not only in physiological cell death concerning generation or differentiation and turn over of normal tissues and cells, but also in some conditions or diseases such as nerve cell death by ischemia after cerebral infarction, cell death caused by radioisotopes or anti-cancer agents, cell death caused by toxins or virus infection, lymphocytopenia due to virus infection such as AIDS, autoimmune diseases and Alzheimer's disease.

In addition to apoptosis, oxidative stress has been implicated in a variety of diseases and pathological conditions. Persistent oxidant damage caused by increased production of free radical species is characteristic in the development of several pathologies, such as neurodegenerative diseases. Practico, et al. report that increased levels of lipid peroxidation (oxidative stress) may be involved in the pathogenesis of Alzheimer's disease². Polyphenolic compounds have been reported to be anti-carcinogenics, anti-inflammatories, and anti-oxidants.

Polyphenolic compounds are bioactive substances that are derived from a variety of plant materials. Polyphenols are a diverse group of compounds which widely arises in a variety of plants, some of which enter into the food chain. In some cases they represent an important class of compounds for the human diet. Although some of the polyphenols are not considered to be nutritious, interest in these compounds has arisen because of their possible beneficial effects on health. These compounds are closely associated with the sensory and nutritional quality of produce derived from these plant materials. Polyphenols are also known to complex with proteins, alkaloids, metal cations, and carbohydrates.

Curcumin (1,7-bis[4-Hydroxy-3-methoxyphenyl]-1,6-heptadiene-3,5-dione) is a member of the class of polyphenolic compounds. It is a yellow spice extracted from the rhizome of Curcuma longa Linn (Zingiberacee), a perennial herb widely cultivated in Asia. It is commonly used as a flavoring and coloring agent in food³. Curcumin is a major active component of turmeric. It contains two electrophilic αβ-unsaturated carbonyl groups, which can react with nucleophiles, such as glutathione. Its anti-inflammatory properties and cancer-preventive activities have been consistently reported using in vitro and in vivo models of tumor initiation and promotion^{4,5}. By virtue of the Michael reaction acceptor functionalities and its electrophilic characteristics, curcumin and several other structurally related polyphenolic compounds have been recently shown to induce the activities of phase II detoxification enzymes, which may be crucial in protecting against carcinogenesis⁶.

Caffeic acid phenethyl ester (CAPE) is an active component of polyphenols derived from the bark of conifer trees and carried by honeybees to their hives. The similarity of CAPE to curcumin is striking because CAPE is also a Michael reaction acceptor that has a broad spectrum of biological activities, including anti-inflammatory^{7,8}, anti-oxidant, and anti-cancer effects^{9,10}.

There is a need for compounds and methods that reduce or prevent damage to cells and tissues, preferably cells of the central and peripheral nervous system, which may occur directly or indirectly as a result of injury, damage, apoptosis, aging, or neurodegenerative disease. One object of the present invention is to fulfill these needs and provide other related advantages. Those skilled in the art will recognize further advantages and benefits of the invention based upon the below-described specification.

### SUMMARY OF THE INVENTION

The present invention relates to protecting cells of the central and peripheral nervous system or tissues from cell death by the administration of an active substance, such as a polyphenolic compound, analog, derivative, or variant thereof, to a subject in need.

One embodiment of the invention relates to a method of protecting neuronal cells, central and peripheral nervous system cells, and cells associated therefrom, from cell death, or apoptosis. The method comprises administering an effective amount of a polyphenolic compound, or analog thereof, sufficient to protect the central and peripheral nervous system cells from dying. Apoptosis may be induced by cell trauma, injury, neurodegenerative disease, or aging.

In another embodiment of the present invention, a method of inducing the activity and expression of proteins which protect neuronal cells, central and peripheral nervous system cells, and cells associated therefrom, or tissues thereof, in a subject from oxidative damage is provided. The method comprises the steps of administering an effective amount of a polyphenolic compound, or analog thereof, sufficient to induce the activity and expression of proteins which protect cells of the central and peripheral nervous system or tissues from oxidative damage. The proteins which protect cells and tissues of the central and peripheral nervous system include, but are not limited to, heme oxygenase-1 (HO-1) and heat shock protein 70 (hsp70).

A further embodiment of the invention relates to the prophylaxis and treatment of degenerative diseases of central and peripheral nervous system tissues in a subject in need thereof, by the administration of an active substance, such as a polyphenolic compound, derivative, analog, and variant thereof, in an amount sufficient to prevent, reduce, or ameliorate the degenerative disease in cells or tissues of the central and peripheral nervous system. Examples of such diseases include neurodegenerative diseases, such as, Parkinson's disease, Alzheimer's disease, HIV dementia, and head and spinal injury.

### BRIEF DESCRIPTION OF THE DRAWINGS

FIG. 1 shows the structure of curcumin and a related analog caffeic acid phenethyl ester (CAPE).

FIG. 2 shows the effect of curcumin on oligonucleosome formation in cerebellar granule cells grown in the absence of (A) serum or (B) low potassium (K+).

FIG. 3 shows the effect of curcumin on oligonucleosome formation in cortical neurons exposed to beta-amyloid peptide (1-40) 20 mM for 48h.

FIG. 4 shows the effect of curcumin on glucose oxidase (GOX) mediated cellular injury in cortical neurons.

FIG. 5 shows the effect of CAPE on oligonucleosome formation in cerebellar granule cells grown in the absence of (A) serum or (B) low potassium (K+).

FIG. 6 shows the effect of CAPE on oligonucleosome formation in cortical neurons exposed to beta-amyloid peptide (1-40) 20 mM for 48h.

FIG. 7 shows the effect of curcumin on (A) heme oxygenase activity; (B) HO-1 protein expression; and (C) hsp70 protein expression in cortical neurons.

FIG. 8 shows the effect of curcumin on mRNA expression (A). The RT-PCR was performed using specific HO-1 primers (B).

FIG. 9 shows the effect of CAPE on (A) heme oxygenase activity; and (B) HO-1 protein expression in cortical neurons.

FIG. 10 shows the relation between HO-1 expression and curcumin neuroprotective effects (A) on a model of neuronal apoptosis by beta-amyloid; and (B) on glucose GOX-mediated cellular injury in cortical neurons.

FIG. 11 shows (A) the protective effects of curcumin in rat models of cerebral neurodegeneration induced by T-butylhydroperoxide (T-BuOOH) and (B) the results of lipid peroxide analysis in different regions of the brain (cortex, striatum, hippocampus, and cerebellum) in curcumin pre-treated rats (T-BuOOH+curcumin), and negative control.

FIG. 12 shows (A) the protective effects of CAPE in rat models of cerebral neurodegeneration induced by T-butylhydroperoxide (T-BuOOH) and (B) the results of lipid peroxide analysis in different regions of the brain (cortex, striatum, hippocampus, and cerebellum) in CAPE pre-treated rats (T-BuOOH+CAPE), and negative control.

FIG. 13 shows the effect of curcumin (A) on heme oxygenase activity and on HO-1 protein expression in astrocytes (B) after short 6 h exposure, and (C) after 24 h exposure at various concentrations of curcumin. Control groups are represented by cells incubated with complete medium alone (0 μM). Each bar represents the mean ±S.E.M. of five independent experiments. *, p<0.05 versus 0 μM curcumin; †,p<0.05 versus 6 h.

FIG. 14 shows the effect of CAPE (A) on heme oxygenase activity and on HO-1 protein expression in astrocytes (B) after a short 6 h exposure, and (C) after a prolonged 24 h exposure at various concentrations of CAPE. Control groups are represented by cells incubated with medium alone (0. μM). Each bar represents the mean ±S.E.M. of five independent experiments. *, p<0.05 versus 0 μM CAPE; †, p<0.05 versus 15, 30, and 50 μM CAPE.

FIG. 15 shows a comparison between the potency of curcumin (CUR) and Curcumin-95 (CUR-95) as inducers of heme oxygenase. Confluent astrocytes were incubated for 6 or 24 h in the presence of various concentrations (15, 30, and 50 μM) of pure curcumin or Curcumin-95 which consists of a mixture of curcuminoids. Each bar represents the mean ±S.E.M. of five independent experiments. *, p<0.05 versus 0 μM Curcumin; †, p<0.05 versus CUR.

FIG. 16 shows the effect of curcumin on intracellular glutathione levels levels. GSH and GSSG levels were measured after (A) 6 hours; or (B) 24 hours exposure of astrocytes to curcumin (0-100 μM). The change in GSH and GSSG levels represent an index of the cellular redox status. Each bar represents the mean ±S.E.M. of four to five independent experiments. *, p<0.05 versus 0 μM.

FIG. 17 shows the effect of CAPE on intracellular glutathione levels. GSH and GSSG levels were measured after (A) 6 hours; or (B) 24 hours exposure of astrocytes to CAPE (0-50 μM). The change in GSH and GSSG levels represents an index of the cellular redox status. Each bar represents the mean ±S.E.M. of four to five independent experiments. *, p<0.05 versus 0 μM.

FIG. 18 shows the effect of N-acetyl-L-cysteine on curcumin-mediated heme oxygenase activation. Astrocytes were exposed to 15, 30, and 50 μM curcumin (CUR) for 6 h in the presence of NAC (1 mM). Each bar represents the mean ±S.E.M. of five independent experiments. *, p<0.01 versus Control (CON); †, p<0.01 versus 30 μM CUR plus NAC.

FIG. 19 shows the effect of curcumin and CAPE on cell viability. Astrocytes were exposed for 24 h to various concentrations (0-100 μM) of (A) curcumin or (B) CAPE in complete medium with and without 1 mM NAC. Data are expressed as the mean ±S.E.M. of six independent experiments. *, p<0.05 versus 0 μM; †, p<0.05 versus curcumin or CAPE alone.

FIG. 20 shows the effect of Curcumin-95 on cell viability. Astrocytes were exposed for 24 h to various concentrations (0-1 00μM) of Curcumin-95 in complete medium. Data are expressed as the mean ±S.E.M. of six independent experiments. *, p<0.05 versus 0 μM.

### DETAILED DESCRIPTION OF THE INVENTION

The present invention pertains to methods of using polyphenolic compounds, analogs, derivatives, or variants thereof, where such compounds are useful for preventing and treating cell death which results from degenerative diseases, disorders, and conditions, apoptosis, cell trauma, injury, neurodegenerative diseases, or aging. As such, the polyphenolic compounds may prevent or treat central and peripheral nervous system tissue damage resulting from cell damage or death due to necrosis or apoptosis, or neurodegenerative diseases in a subject, preferably a mammal, and more preferably a human.

The invention is based on the finding that polyphenolic compounds prevent or reduce the process of programmed cell death known as apoptosis. Apoptosis may be actively triggered in cells by, for example, exposure to X-radiation, cytotoxic drugs, free-radicals and heat, or it may be unmasked by removal of critical peptide growth factors, steroid hormones, lymphokines or neurotrophins that constantly suppress programmed cell death in various tissues. Many of these processes are the terminal events involved in numerous disease states or the final events by which therapeutic treatments effect their results. Thus, specifically targeting and altering apoptosis provides a general treatment for a broad range of diseases and pathological conditions associated with apoptosis, including neurodegenerative diseases or disorders.

The polyphenolic compounds used in the present invention include but are not limited to natural plant extracts such as curcumin (1,7-bis[4-Hydroxy-3-methoxyphenyl]-1,6-heptadiene-3,5-dione) and caffeic acid phenethyl ester (CAPE), polyphenolic compound analogs, derivatives, or variants thereof, and optionally, a combination of polyphenols or other therapeutic agents useful in preventing or treating cell death or a disease associated therewith. The polyphenols useful in the present invention are those that are similar in chemical structure to curcumin (1,7-bis[4-Hydroxy-3-methoxyphenyl]-1,6-heptadiene-3,5-dione) or CAPE, and retain Michael reaction acceptor functionalities. In one embodiment, the polyphenols are natural and derived from plant materials. Another embodiment encompasses natural or synthetic polyphenols having a similar chemical structure to curcumin and/or CAPE.

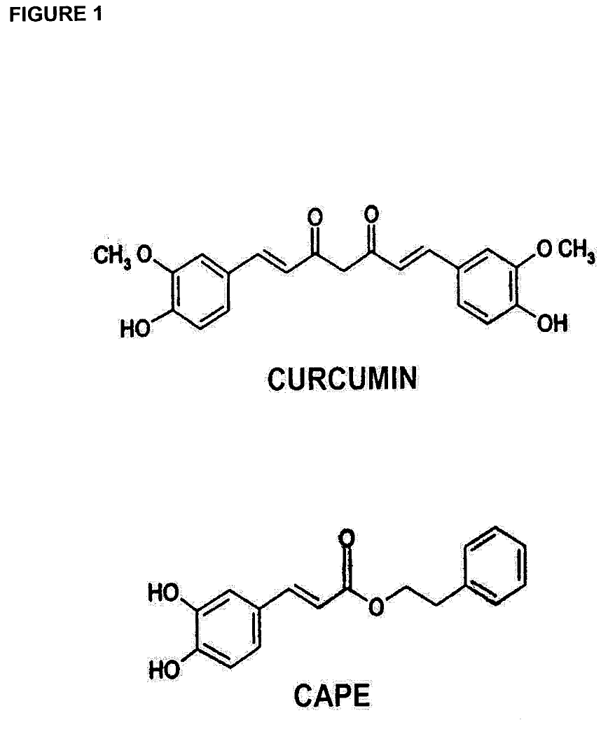

Analogous compounds include, but are not limited to ester, dimeric ether, and other chemical synthetic compounds related to the curcumin (1 ,7-bis[4-Hydroxy-3-methoxyphenyl]-1,6-heptadiene-3,5-dione) or CAPE chemical structure as shown in FIG. 1. In particular, other derivatives from Curcuma Longa, such as demethoxycurcumin and bisdemethoxycurcumin or any polyphenol with modifications in one of the methoxyl groups from the molecule of curcumin or CAPE. Generally, electrophilic polyphenols having Michael reaction acceptor activity and capable of specifically inducing HO-1 and/ or Hsp70 are provided in this invention. The preferred polyphenolic compounds are natural, non-toxic, and safe for human use.

In one embodiment of the invention, a method of protecting neuronal cells, central and peripheral nervous system cells, and cells associated therefrom, from cell death or apoptosis, as well as diseases associated with cell death or apoptosis, is provided, where an effective amount of polyphenolic compound, or analog thereof, is administered to a subject in need thereof, sufficient to protect neuronal cells, central and peripheral nervous system cells, and cells associated therefrom, from cell death. Neuronal cells include, but are not limited to any of the conducting cells of the central and peripheral nervous system. A further embodiment of this invention also provides protection of cerebral non-neuronal cells, such as glial cells and specific brain endothelial cells.

Another embodiment of the present invention relates to polyphenolic compounds useful for protecting neuronal cells, central and peripheral nervous system cells, and cells associated therefrom, against apoptosis and cellular stresses. Natural, isolated polyphenolic compounds, or analogs thereof, showing apoptotic inhibitory activity may be used as an agent for prophylaxis and treatment of diseases which are thought to be mediated by the promotion of apoptosis, such as viral diseases, neurodegenerative diseases, myelodysplasis, ischemic diseases and hepatic diseases. Neurodegenerative disorders including ALS and Parkinson's disease are characterized by progressive neuronal cell death. Apoptosis, a morphologically and biochemically defined form of cell death caused by active cellular signaling, has long been implicated in neurodegeneration. The use of anti-apoptotic therapies for neurodegenerative disorders has not previously been successful, particularly because many of the compounds have high levels of toxicity. Moreover, interfering with apoptotic pathways has often resulted in an augmented risk of disease.

Accordingly, one embodiment of the invention relates to a method of protecting neuronal cells, central and peripheral nervous system cells, and cells associated therefrom, from cell death or apoptosis, where a polyphenolic compound such as but not limited to, curcumin, CAPE or an analog thereof, is administered to a subject in need thereof, in an effective amount such that the neuronal cells, central and peripheral nervous system cells, and cells associated therefrom, are protected from cell death or apoptosis.

Thus, in another embodiment of the invention, polyphenolic compounds, or analogs thereof, that protect neuronal cells, central and peripheral nervous system cells, and cells associated therefrom, and impair apoptosis are used in methods to inhibit disease-induced apoptosis or to selectively enhance neuroprotective proteins by administering to a subject in need thereof, an effective amount of a polyphenolic compound, or analog or variant thereof, to prevent or treat disease-induced apoptosis, or to selectively enhance neuroprotective proteins in the subject. The disease is preferably a neurodegenerative disease, disorder, or condition.

Prophylactic neuroprotection may be administered to populations at high-risk for cell death of neuronal cells, cells of the central and peripheral nervous system, and those associated thereof. These would include (1) short term neuroprotection both prior to and after high-risk invasive procedures whose adverse event produce injury or death to neuronal cells, cells of the central and peripheral nervous system, and those associated thereof; and (2) chronic neuroprotection for high-risk populations with systemic disease or multiple risk factors which increase the probability of cell injury or death to neuronal cells, cells of the central and peripheral nervous system, and those associated thereof. One such example is a person having a family medical history of a neurodegenerative disease. Proteins which protect neuronal cells, central and peripheral nervous system cells, and cells associated therefrom, and tissues are mediators of apoptotic events, thus agents that modulate these neuroprotective proteins are useful for treating or preventing neurodegenerative diseases, disorders, or conditions. Modulators of these proteins provide a useful therapeutic for treating conditions involving cell death, and also for preventing such neurodegenerative conditions.

Well-established paradigms of programmed cell death in cerebral granule cells and cortical neurons may be used to test the effects of polyphenolic compounds useful in neuroprotection and anti-apoptosis. Examples of such tests include depriving cerebral granule cells of serum or the use of a low concentration of potassium (low K⁺) and exposing cortical neurons to β-amyloid peptide, a known inducer of apoptosis. One means for measuring apoptosis is the use of an immunodetection assay that measures oligonucleosome formation. Viability of cortical neurons may also be determined in order to assess the neuroprotective activity of polyphenolic compounds.

In another embodiment of the invention, a method of reducing cell injury, damage, or cell death (neuronal apoptosis) of neuronal cells, central and peripheral nervous system cells, and cells associated therefrom, induced in neurodegenerative diseases or disorders, including aging, comprises the administration of a therapeutically effective amount of a polyphenolic compound, or analog thereof, to a subject in need of such therapy.

The present invention is also based on the finding that curcumin is an anti-oxidant and anti-inflammatory that induces heme oxygenase-1 and protects endothelial cells against oxidative stress¹¹. Curcumin is a member of the polyphenol class, as is caffeic acid phenethyl ester (CAPE). CAPE, another plant-derived polyphenolic compound, has been shown to increase heme oxygenase activity and HO-1 protein expression in astrocytes¹².

Polyphenolic compounds have an enormous range of biological activity and are known to inhibit oxidative damage in vivo better than the classical vitamin anti-oxidants. In plants, polyphenols protect against lipid peroxidation and UV damage that can affect tropical fruits growing under severe conditions including high heat and intense sunlight. Stress proteins have been implicated in playing a role in maintaining cellular homeostasis. Accordingly, polyphenolic compounds may be useful in cellular homeostasis, modulating the activity of stress proteins, and in preventing or treating neurodegenerative diseases and disorders. Since a balance between cellular formation and apoptosis is critical for maintaining cellular homeostasis, the invention provides methods of preventing or treating diseases associated with cell death or apoptosis as a result of oxidative damage sufficient to reduce or ameliorate cell death using agents, such as polyphenolic compounds, or analogs thereof.

Stress proteins have been shown to be associated with cellular homeostasis. Among the molecules belonging to the stress protein family, two inducible proteins have been particularly studied for their potential role in protecting neurons against cell death. These neuroprotective proteins are heme oxygenase 1 (HO-1) and heat shock protein 70 (hsp 70). In the brain, the heme oxygenase system has been reported to be very active and its modulation seems to play a crucial role in the pathogenesis of neurodegenerative disorders.

HO-1 is a ubiquitous and redox-sensitive inducible stress protein. Heme is a substrate for HO-1 in the formation of carbon monoxide, free ferrous iron, and biliverdin, where biliverdin is quickly converted to bilirubin by biliverdin reductase. HO-1 may play an important role in the central nervous system. Activation of HO-1 offers an important defensive mechanism for neurons exposed to oxidative stress or damage. Chen, et al. demonstrate that activation of HO-1 in neurons is strongly protective against oxidative damage¹³. The second protein, heat shock protein 70 (hsp70) is a fundamental protein used by the cells to refold mutated proteins and to maintain internal homeostasis. Hsp70 activity may be related to the regulation of apoptosis in neurons. Plant-derived natural substances, such as polyphenolic compounds, that are non-toxic, safe for human use, and trigger HO-1 and hsp70 expression and other intracellular defense systems, therefore, clearly offer a great advantage for therapeutic purposes.

One embodiment of the invention relates to a method of inducing neuroprotective protein activity and expression. A related embodiment encompasses the administration of a modulator, preferably an agonist or activator, of a neuroprotective protein, in an amount effective to treat, reduce, and/or ameliorate oxidative damage, or the symptoms incurred thereof.

An “agonist” refers to a molecule which, when bound to, or associated with a neuroprotective protein or a functional fragment thereof, increases or prolongs the duration of the effect of the neuroprotective protein or polypeptide. Agonists may include proteins, nucleic acids, carbohydrates, or any other molecules that bind to and modulate the effect of the neuroprotective protein or polypeptide. Agonists typically enhance, increase, or augment the function or activity of the neuroprotective protein. Such neuroprotective proteins may include, but are not limited to, cellular stress response-related proteins, heme oxygenase 1 (HO-1) and heat shock protein 70 (hsp70). The agonist or activator is preferably a polyphenolic compound, such as but not limited to, curcumin or CAPE, or an analog, derivative or variation thereof.

An “antagonist” refers to a molecule which, when bound to, or associated with, a protein associated with apoptosis, or a functional fragment thereof, decreases the amount or duration of the biological activity of the apoptotic protein. Antagonists may include proteins, nucleic acids, carbohydrates, antibodies, or any other molecules that decrease or reduce the effects of apoptosis or cell death.

A further embodiment of the invention encompasses a method of treating neurodegenerative diseases, disorders or conditions comprising the administration of a therapeutically effective amount of a polyphenolic compound, or analog thereof, to a subject in need of such therapy. In a preferred embodiment, the subject is mammalian, more preferably human.

Non-limiting examples of neurodegenerative diseases, disorders, and conditions include neurological disorders related to excessive activation of excitatory amino acid receptors or the generation of free radicals in the brain which cause nitrosative or oxidative stress, including aging, stroke (e.g., cerebral ischemia and hypoxia ischemia), hypoglycemia, domoic acid poisoning (from contaminated mussels), anoxia, carbon monoxide or manganese or cyanide poisoning, central nervous system infections such as meningitis, dementia (particularly HIV-mediated dementia) and neurodegenerative diseases such as Huntington's disease, Alzheimer's disease, Parkinson's disease, head and spinal cord trauma, epilepsy (e.g., seizures and convulsions), olivopontocerebellar atrophy, amyotrophic lateral sclerosis (ALS), meningitis, multiple sclerosis and other demyelinating diseases, neuropathic pain (painful peripheral neuropathy, such as diabetic neuropathy and HIV-related neuropathy), mitochondrial diseases (e.g., MERRF and MELAS syndromes, Leber's disease, Wernicke's encephalophathy, Rett syndrome, homocysteinuria, hyperprolinemia, hyperhomocysteinemia, nonketotic hyperglycinemia, hydroxybutyric aminoaciduria, sulfite oxidase deficiency, combined systems disease, and lead encephalopathy), Tourette's syndrome, hepatic encephalopathy, drug addiction, drug tolerance, drug dependency, depression, anxiety, and schizophrenia.

Another embodiment of the present invention relates to pharmaceutical or physiological compositions, preferably containing a pharmaceutically or physiologically acceptable vehicle, such as a carrier, diluent, or excipient. According to the invention, pharmaceutical or physiological compositions comprise one or more polyphenolic compounds, or an analog thereof, or a pharmaceutically acceptable salt, either alone or in combination with a biologically active agent, such as drugs, steroids, or synthetic compounds, particularly for use in the methods according to the present invention, and a pharmaceutically or physiologically acceptable vehicle. The term “pharmaceutically acceptable salts” refers to salts prepared from pharmaceutically acceptable non-toxic acids and bases, including inorganic and organic acids and bases. The pharmaceutical compositions may preferably comprise the polyphenolic compound, or analog thereof, of the present invention, such as for example, curcumin or CAPE. Such a pharmaceutical or physiological composition may be administered to any subject in need of such therapy, including, for example, mammals such as monkeys, dogs, cats, cows, horses, rabbits, and most preferably, humans, for any of the above-described therapeutic or preventative uses and effects.

The pharmaceutical compositions include compositions suitable for oral and parenteral (including subcutaneous, intramuscular, intrathecal, intravenous, and other injectables) routes, although the most suitable route in any given case will depend on the nature and severity of the condition being treated.

In addition, the pharmaceutically acceptable vehicle may be delivered via charged and uncharged matrices used as drug delivery devices such as cellulose acetate membranes, also through targeted delivery systems such as liposomes attached to antibodies or specific antigens.

In practical use, polyphenols, or analogs thereof, may be combined as the active ingredient(s) in intimate admixture with a pharmaceutical carrier according to conventional pharmaceutical compounding techniques. The carrier may take a wide variety of forms depending on the form of preparation desired for administration, e.g., oral or parenteral (including tablets, capsules, powders, intravenous injections or infusions).

In preparing the compositions of the present invention for oral dosage form any of the usual pharmaceutical media may be employed, e.g., water, glycols, oils, alcohols, flavoring agents, preservatives, coloring agents, and the like; in the case of oral liquid preparations, e.g., suspensions, solutions, elixirs, liposomes and aerosols; starches, sugars, micro-crystalline cellulose, diluents, granulating agents, lubricants, binders, disintegrating agents, and the like in the case of oral solid preparations e.g., powders, capsules, and tablets. In preparing the compositions for parenteral dosage form, such as intravenous injection or infusion, similar pharmaceutical media may be employed, e.g., water, glycols, oils, buffers, sugar, preservatives and the like know to those skilled in the art.

In the methods of the present invention, polyphenolic compounds, or analogs thereof, or pharmaceutical or physiological compositions thereof, can be administered alone or in combination with at least one other biologically active agent, which may be introduced in any sterile, biologically compatible pharmaceutical or physiologically acceptable carrier, excipient, or diluent, including, but not limited to, saline, buffered saline, dextrose, and water. The compositions may be administered to a patient alone, or optionally in combination with other biologically active agents, drugs, or hormones.

In addition, in the methods according to this invention, the polyphenolic compounds, or analogs thereof, or pharmaceutical or physiological compositions thereof, may be administered by any number of routes including, but not limited to, oral, nasal, intravenous, intramuscular, intra-arterial, intramedullary, intrathecal, intraventricular, transdermal, subcutaneous, intraperitoneal, intranasal, enteral, topical, and sublingual means. Preferably, the polyphenolic compounds, or analogs thereof, are administered orally, nasally, or by inhalation. Administration of polyphenolic compound compositions of the invention may also include local or systemic administration, including injection, oral administration, particle gun, or catheterized administration, and topical administration. Various methods may be used to administer a polyphenolic composition directly to a specific site in the body. For example, in instances where topical delivery is preferred, transdermal patches and/or permeation enhancers may be used, a variety of which are commonly known in the art.

Both the dose of a polyphenolic compound, or analog thereof, or composition thereof, and the means of administration may be determined based on the specific qualities of the polyphenolic compound or therapeutic composition thereof; the condition, age, and weight of the patient; the progression of the disease; and other relevant factors. Preferably, a polyphenolic compound, or analog thereof, or therapeutic composition thereof, according to the invention, increases the activity and expression of neuroprotective proteins or decreases cell death or apoptosis.

In addition to the active ingredients of the invention, i.e., polyphenolic compounds, or analogs thereof, or compositions thereof, the pharmaceutical compositions may contain suitable pharmaceutically acceptable carriers, diluents, or excipients comprising auxiliaries which facilitate processing of the active compounds into preparations which may be used pharmaceutically. Further details on techniques for formulation and administration are provided in the latest edition of Remington's Pharmaceutical Sciences (Mack Publishing Co.; Easton, Pa.).

Pharmaceutical compositions for oral administration may be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use may be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/ or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which may be orally administered in the methods according to the present invention, include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration in the methods of the present invention may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants or permeation-enhancing agents that are appropriate to the particular barrier to be permeated are used in the formulation. Such penetrants are generally known in the art.

The pharmaceutical compositions of the present invention, comprising a polyphenolic compound, or analog thereof, may be manufactured in a manner that is known in the art, e.g., by means of conventional mixing, dissolving, granulating, dragee-making, levigating, emulsifying, encapsulating, entrapping, or lyophilizing processes.

In addition to the formulations or compositions described previously, the polyphenolic compounds, or analogs thereof, may also be formulated as a sustained and/or timed release formulation. Such sustained and/or timed release formulations may be administered by implantation (for example, subcutaneously or intramuscularly) or by intramuscular injection. Thus, for example, the polyphenolic compounds, or analogs thereof, may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs. Common timed and/or controlled release delivery systems include, but are not be restricted to, starches, osmotic pumps, or gelatin micro capsules.

The magnitude of a therapeutic dose of polyphenolic compounds, or analogs thereof, in the acute or chronic management of neurodegenerative diseases may vary with the severity of the condition to be treated and the route of administration. The dose, and dose frequency, will also vary according to the age, body weight, condition and response of the individual patient, and the particular polyphenolic combination used. All combinations described in the specification are encompassed as therapeutic, active polyphenol mixtures and it is understood that one of skill in the art would be able to determine a proper dosage of particular polyphenol mixtures using the parameters provided in the invention. Generally, the daily dose of the active polyphenol ranges from about 200 milligrams to about 500 milligrams administered orally.

For example, in one embodiment, the daily dose ranges of a polyphenolic compound, or analog thereof, such as curcumin or CAPE, for the conditions described herein are generally from about 3 mg to about 10 mg per kg body weight of the polyphenol, preferably a daily dose of 5-7 mg/kg. The polyphenol formulation of the invention is preferably given two times per day, once in the morning and once in the evening, totaling 200-500 mg when administered orally. When the dose is administered orally, a sustained release formulation is may be used so that a fairly constant level of polyphenols is provided over the course of treatment, which is generally at least 48 hours and preferably at least 96 hours per cycle. As the polyphenols are not toxic, the formulation may be administered for as long as necessary to achieve the desired therapeutic effect.

Polyphenolic compounds may be combined in a single formulation for preventing or treating neurodegenerative diseases or cell death. The compounds are present in varying percentages in the formulation. Thus, the formulation may be adjusted to reflect the concentrations of polyphenolic compounds.

In an alternative embodiment of the invention, the effect of the therapy with polyphenolic compounds on cell death treatment may be monitored by any methods known in the art, including but not limited to monitoring heme oxygenase activity or expression in patient sera, as well as more traditional approaches such as determining levels of oligonucleosome formation and glucose oxidase (GOX)-mediated cellular injury, and changes in morphology and/or size using computed tomographic scans.

Desirable blood levels may be maintained by a continuous infusion of polyphenols as ascertained by plasma levels. It should be noted that the attending physician will also know how to and when to adjust treatment to higher levels if the clinical response is not adequate (precluding toxic side effects, if any).

Again, any suitable route of administration may be employed for providing the patient with an effective dosage of curcumin or CAPE, or analogs thereof, or a polyphenol combination of this invention. Dosage forms include tablets, troches, cachet, dispersions, suspensions, solutions, capsules, gel caps, caplets, compressed tablets, sustained release devices, patches, and the like.

The following examples illustrate production and use of the present invention. These examples are offered by way of illustration, and are not intended to limit the scope of the invention in any manner. All references described herein are expressly incorporated in toto by reference.

### EXAMPLES

### Example 1

Neuroprotective and Anti-Apoptotic Effects of Polyphenolic Compounds

Primary cultures of cortical neurons and cerebellar granule cells were prepared. In brief, cortical neurons were prepared from fetal brain from 17-day pregnant rat and cerebellar granule cells from 8-day old rats. Brain tissues were dissociated through trypsinization in 0.025% trypsin solution and trituration. Dissociated cells were collected through centrifugation and resuspended in standard medium containing: basal Eagle's medium containing 10% fetal bovine serum (FCS), 2 mM glutamine, genatmycin 0.05 mg/ml, and 25 mM KCI. Cells were plated at a density of 1.8×10⁶ onto 35-mm dishes coated with 10 micrograms/ml ply-L-lysine.

To induce programmed cell death in cerebral granule cells, two different experimental well-established paradigms: serum deprivation and low K⁺, were performed. In particular, neurons were cultured after their plating in serum free medium containing 25 mM KCI or in standard medium for 6 days and then agitating them in a medium containing 10% FCS and 5 mM KCI. In both of these paradigms, control cells were grown in medium containing 10% FCS and 25 mM KCI.

To induce apoptosis in cortical neurons, β-amyloid peptide (1-40), a molecule related to the pathogenesis of Alzheimer disease and a well known inducer of apoptosis was added. The neurons after 6 days were treated with 20 μM of (β-amyloid peptide (1-40) or with β-amyloid peptide (40-1) (an inactive form of amyloid that is used for control) at different times. The levels of apoptosis were analyzed by immunodetection of the oligonucleosomes released from the nucleus into the cytoplasm of apoptotic neurons. The sandwich enzyme-linked immunosorbent assay (cell death detection ELISA, Roche) was used.

Curcumin

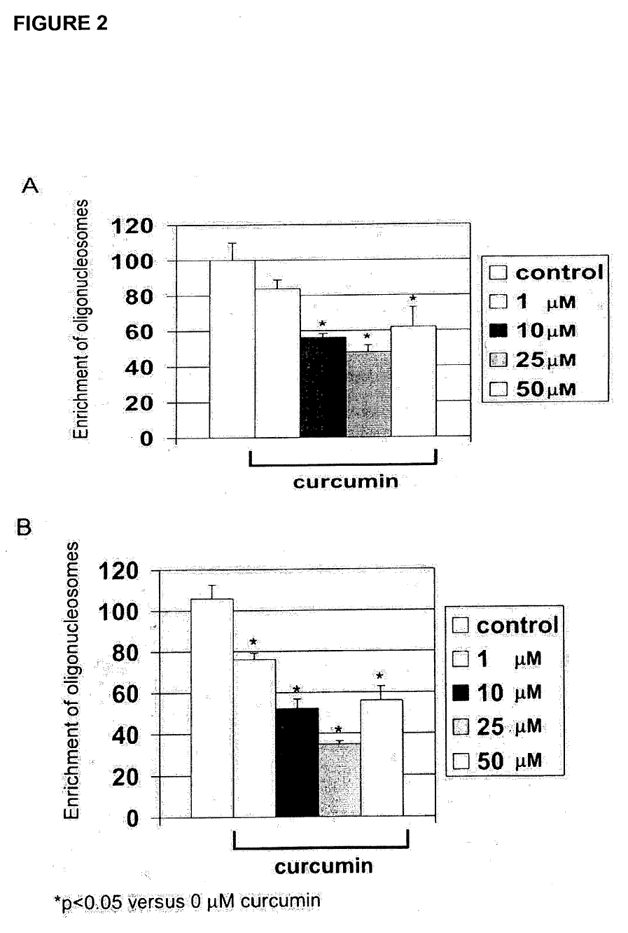

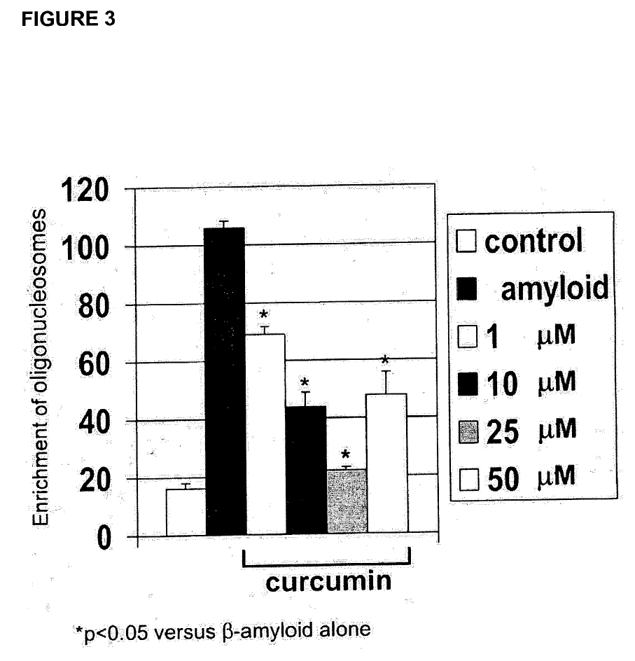

Exposure of cerebellar granule cells to different concentrations of curcumin (1, 5, 10, 25 and 50 μM) significantly reduced apoptosis induced by the absence of FCS (FIG. 2A) and by reducing the concentration of extracellular potassium (K⁺) (FIG. 2B). Specifically, curcumin (25 μM) resulted in the strongest anti-apoptotic effect, particularly in the cells exposed to low K⁺. In cortical neurons exposed to 20 μM of β-amyloid peptide (1-40), a parallel treatment with curcumin was able to dramatically protect the cells against apoptosis (FIG. 3).

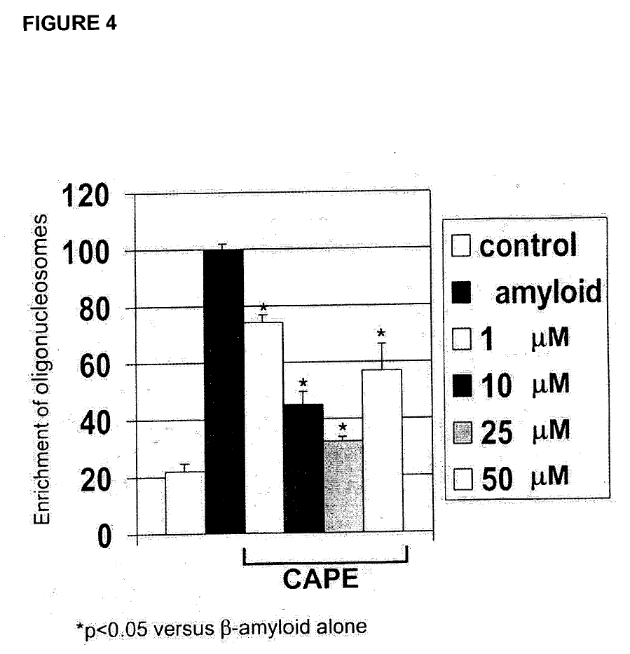

To better assess the neuroprotective activity of curcumin, cell viability was determined for cortical neurons treated with 25 μM curcumin for 12 h followed by incubation for 2 h in the presence of glucose oxidase (100 mU/ml). This oxidant system generates hydrogen peroxide at a constant rate and is known to promote cellular injury in vitro¹⁴. After treatment with glucose oxidase, cells were washed and exposed to complete medium containing 1% Alamar blue for 5 h according to manufacturers' instruction (Serotec, UK), in order to assess cell viability. After the 2 h incubation period, optical density in the medium of each well was measured using a plate reader (Molecular Devices; Crawley, UK). The assay is based on detection of metabolic activity of living cells using a redox indicator, which changes from oxidized (blue) to reduced (red) form. The intensity of the red color is proportional to the viability of cells, which is calculated as a difference in absorbance between 570 nm and 600 nm and expressed as a percentage of control. Treatment of cells for 2 h to glucose oxidase, which generated hydrogen peroxide in the culture medium, resulted in 68% decrease in cell viability (FIG. 4). However, exposure of cells for 24 h to curcumin 25 μM reduced glucose oxidase-mediated damage and restored cell viability to 70% of control.

Caffeic Acid Phenethyl Ester (CAPE)

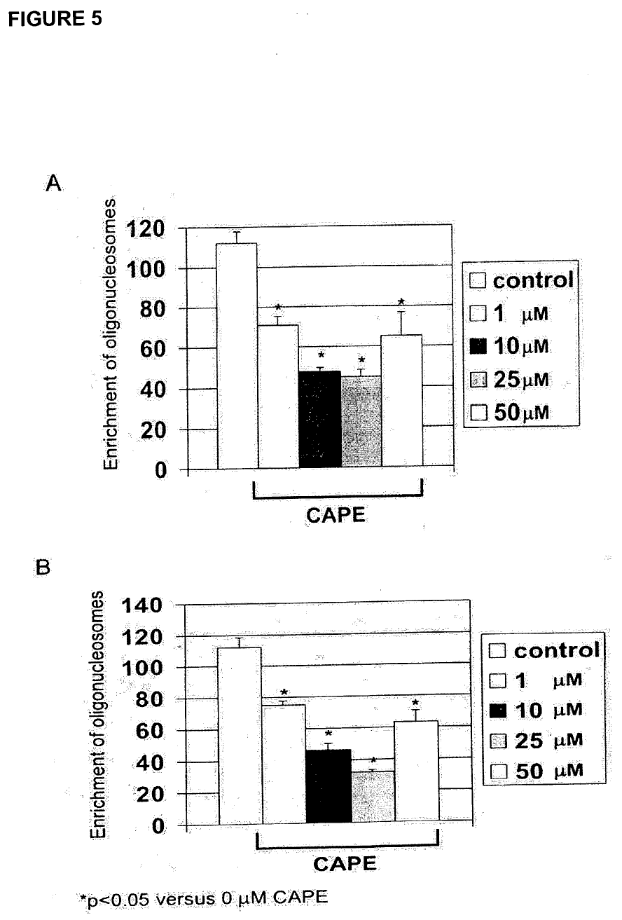

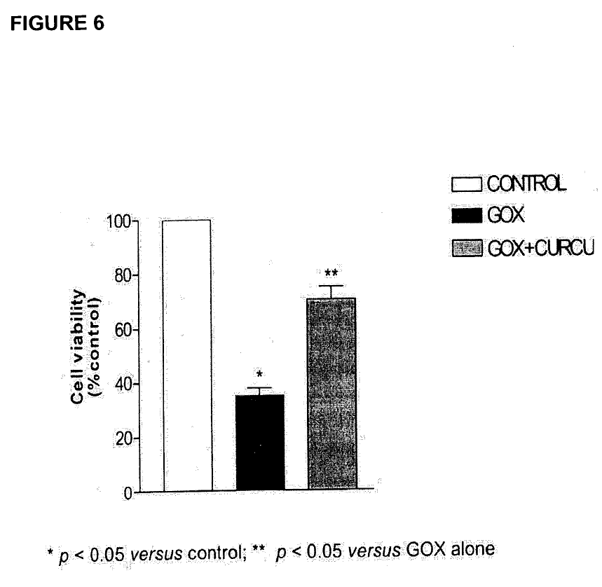

For these studies, cerebellar granule cells were deprived of FSC or exposed to low K⁺ and cortical neurons were exposed to β-amyloid, as previously described. As shown in FIG. 5 and FIG. 6, CAPE had a strong dose-dependent anti-apoptotic effect in all three of the models examined. A concentration of 25 μM was found to be most effective in protecting neuronal cells from apoptosis.

### Example 2

Effects of Polyphenolic Compounds on Heme Oxygenase-1 and HSP70 Activity in Neurons

The ability of curcumin to interact with the heme oxygenase (HO) pathway in primary cortical neurons was tested. Cells were exposed to various concentrations of curcumin (1-100 μM) and heme oxygenase activity. HO-1 mRNA and protein expression were determined at different times after treatment. N-acetyl cysteine (NAC; 1 mM), a precursor of glutathione and a sulphydril donor with potent anti-oxidant properties, was also used to examine whether changes in heme oxygenase activity by curcumin are mediated by pro-oxidant mechanisms.

Heme oxygenase activity assays were performed using a modified methodology previously described by Maines, et al.¹⁵ Cells were washed with PBS, scraped off their plates, separated by centrifugation, and resuspended in a solution containing 100 mM PBS and 2mM MgCl₂, freeze/thawed three times, and finally sonicated. The supernatant was added to a reaction mixture containing nicotinamide adenine dinucleotide phosphate (NADPH) (0.8 mM), glucose 6-phosphate (2 mM), glucose-6-phosphate dehydrogenase (0.2 units), 3 mg of rat liver cytosol prepared from a 105,000×g supernatant fraction as a source of biliverdin reductase, potassium phosphate buffer (PBS, 100 mM, pH 7.4), MgCl₂ (0.2 mM), and hemin (20 μM). The reaction was conducted at 37° C. in the dark for 1 h and terminated by the addition of 1 ml of chloroform. The amount of extracted bilirubin was calculated by the difference in absorbance between 464 nm and 530 nm (ε=40 mM⁻¹ cm⁻¹). Heme oxygenase activity was expressed as picomoles of bilirubin/mg of cell protein/h. The total protein content of confluent cells was determined using a Bio-Rad DC protein assay (Bio-Rad; Herts, UK) by comparison with a standard curve obtained with bovine serum albumin.

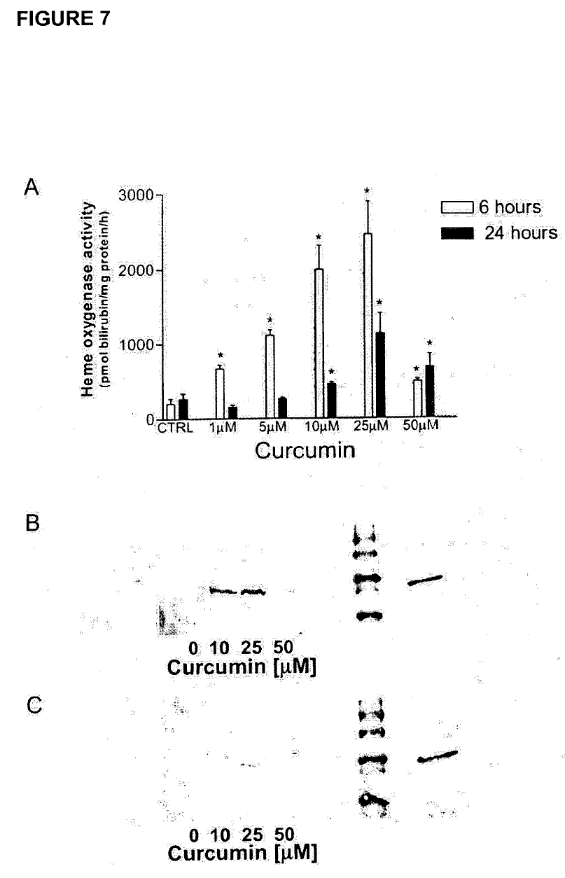

HO-1 and hsp70 protein levels were assessed by Western immunoblot technique using a polyclonal rabbit anti-HO-1 antibody (Stressgen; Victoria, Canada), and a polyclonal rabbit hsp70 antibody. Briefly, an equal amount of proteins (30 μg) for each sample was separated by SDS-polyacrylamide gel electrophoresis and transferred to nitrocellulose membranes, and the non-specific antibodies were blocked with 3% non-fat dried milk in PBS. Membranes were then probed with a polyclonal rabbit anti HO-1 antibody (Stressgen) (1:1000 dilution in Tris-buffered saline, pH 7,4) or with a monoclonal rabbit anti-Hsp72 antibody (Amersham Pharmacia Biotechnology) (1:1000 dilution in Tris-buffered saline, pH 7,4), that recognizes only the Hsp 70 inducible isoform. Blots were then visualized using an amplified alkaline phosphate kit from Sigma. The effects of various concentrations of curcumin (1, 5,10, 25 and 50 μM) on heme oxygenase activity are shown in FIG. 7A. Exposure of neurons to curcumin (1-50 μM) for 6h resulted in a concentration-dependent increase in heme oxygenase activity. The increase was significantly different from controls (untreated cells, p<0.05), with a maximal enzymatic activity (10-fold increase) at 25 μM curcumin. At concentrations of 50 μM curcumin, heme oxygenase activity gradually decreased to those of control values. The dose-dependent induction of heme oxygenase activity by curcumin was maintained after 24h exposure.

Western blot analysis revealed that enhanced heme oxygenase activity by curcumin treatment was directly correlated with HO-1 protein levels (FIG. 7B). Western blot analysis also showed that curcumin treatment induced hsp70 protein expression (FIG. 7C).

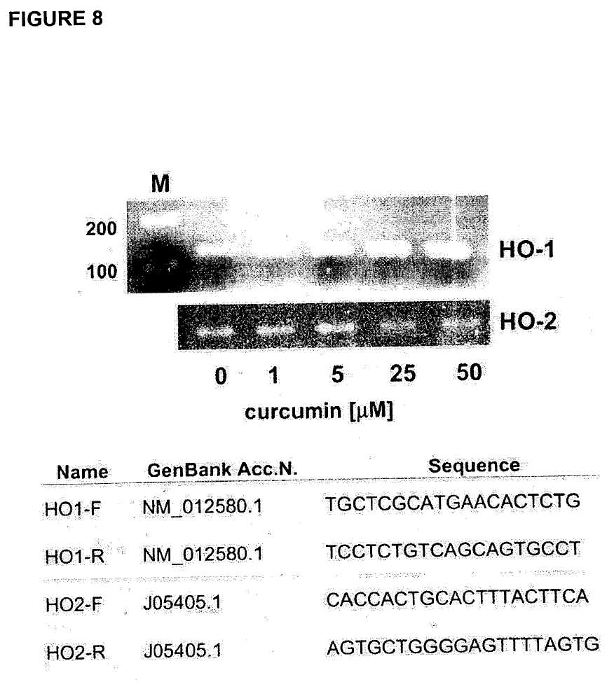

A semi-quantitative RT-PCR was performed to investigate the activity of curcumin on HO-1 mRNA expression, using specific HO-1 primers (FIG. 8) that generated a 123 bp product. To control the integrity of RNA and for differences attributable to errors in manual experimental manipulation, primers for rat heme oxygenase 2 (HO-2), a constitutive gene that does not change its expression, were used in separate PCR reactions. The HO-2 primers generated a 331-bp PCR product. As shown in FIG. 8A, curcumin was able to induce a dose-dependent over-expression of HO-1 mRNA.

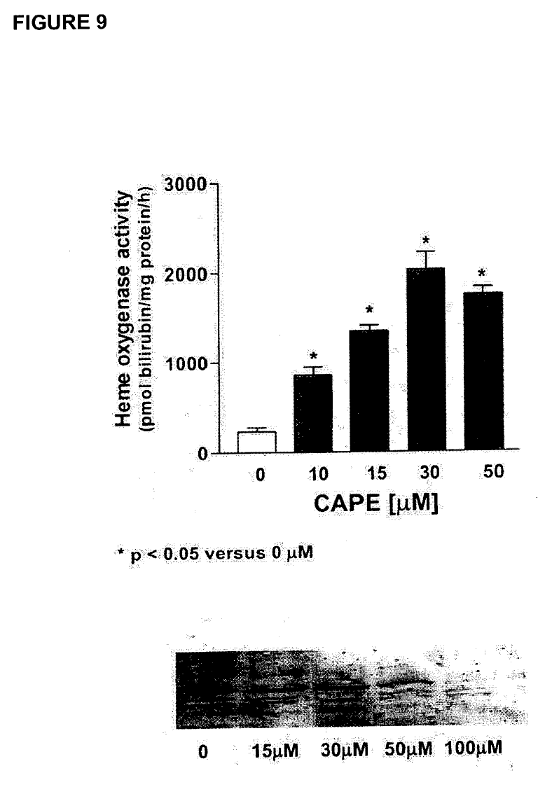

In order to determine whether other molecules within the class of the invention were able to induce HO-1 activity, cultured neurons were exposed to various concentrations of CAPE (1-100 μM ). Heme oxygenase activity and protein expression were determined at different times after polyphenol treatment. As shown in FIG. 9, CAPE was shown to strongly induce HO-1.

In order to test the effects of polyphenolic compounds, or analogs thereof, on heme oxygenase activity, curcumin and CAPE were added to cortical neurons for analysis. Heme oxygenase activities resulting from curcumin and CAPE treatment are shown in FIGS. 7A and 9A, respectively. Neuroprotective protein expression is observed in FIGS. 7B-C and 9B. The activity of curcumin on HO-1 mRNA expression may be measured to ensure that heme oxygenase activity correlates with mRNA expression (FIG. 8). Semi-quantitative reverse transcriptase-polymerase chain reaction using HO-1 and HO-2 (control) forward and reverse primers are as follows:

| PRIMER | | SEQ ID | GENBANK |
|---|---|---|---|
| NAME | SEQUENCE | NO: | ACC. NO. |
| HO1-F | 5′-TGCTCGCATGAACACTCTG-3′ | 1 | NM_012580.1 |
| HO1-R | 5′-TCCTCTGTCAGCAGTGCCT-3′ | 2 | NM_012580.1 |
| HO2-F | 5′-CACCACTGCACTTTACTTCA-3′ | 3 | J05405.1 |
| HO2-R | 5′-AGTGCTGGGGAGTTTTAGTG-3′ | 4 | J05405.1 |

An inhibitor of HO-1 activity may be used to determine whether or not the effects of the polyphenolic compounds or analogs thereof are induced through the heme oxygenase signaling pathway. To understand the relevance of the induction of the stress protein HO-1 on the neuroprotective effects afforded by curcumin we treated cortical neuron exposed to β-amyloid with curcumin (25 mM) alone or in the presence of tinprotoporphirine IX (SnPP) 40 μM, a specific inhibitor of HO-1 activity. The same treatment was performed in cortical neurons exposed to GOX-induced cellular damage.

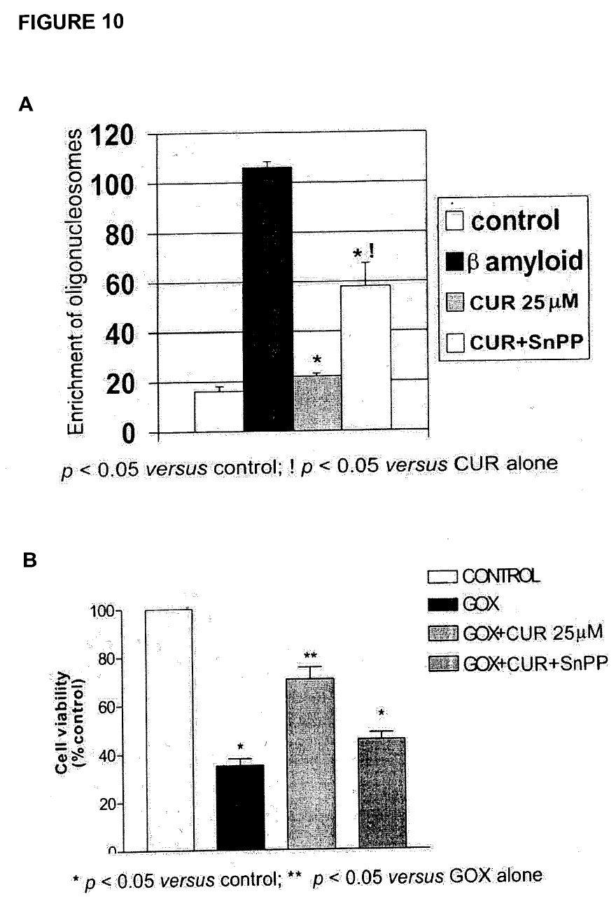

The experiment depicted in FIG. 10 uses SnPP to inhibit HO-1 activity, thereby showing that curcumin is effective through a heme oxygenase-associated signaling pathway. As shown in FIG. 10, the inhibition of HO-1 activity caused by SnPP reduced neuroprotective and anti-apoptotic effects of curcumin. Although curcumin alone was able to overcome much of the cellular damage caused by β-amyloid, curcumin was only able to partially reduce the apoptotic effects caused by the addition of SnPP. Similar results were observed in the cell viability assay using GOX-induced cellular damage, where curcumin was able to overcome the apoptotic effects of GOX alone, but only partially in combination with SnPP. These data support the importance of HO-1 induction in the mechanisms of action induced by curcumin and analogous polyphenols.

### Example 3

Protective Effects of Curcumin in Models of Cerebral Neurodegeneration

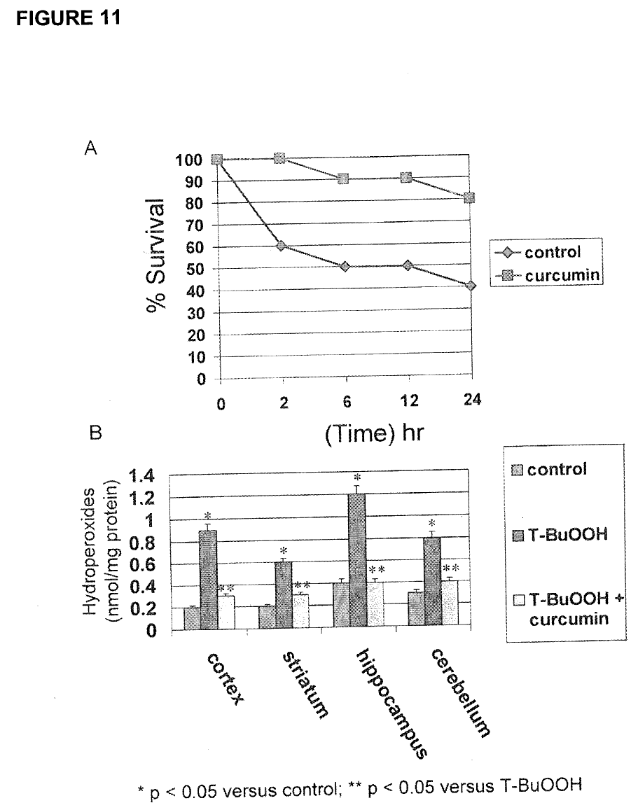

Male Wistar rats, weighing 140-180 g, were used for all experiments. Ten animals per experimental group were pre-administrated with curcumin, suitably suspended in distilled water, at the dose of 50 mg/kg per day, through oral intubation, for 7 days, before an intracerebroventricular injection of T-butylhydroperoxide (T-BuOOH) 2 μl of a 70% solution of phosphate buffered saline (PBS)¹⁶. Lethality was assessed after administration of T-BuOOH at time points: 0, 2, 6, 12, and 24 h, and was expressed as the percentage of survival relative to the lethality observed in T-BuOOH plus Tween/saline vehicle (control) treated animals (FIG. 11A). After 30h the surviving animals were sacrificed and their brains quickly removed to investigate lipid peroxide formation as an index of oxidative challenge in neurons.

Briefly, brain areas were dissected and homogenized in ice cold 0.1 M, pH 7.5 phosphate buffer (final vol 1.1 ml). Aliquots (0.5 ml) of brain homogenates were transferred to a mixture of ice cold water (600 μl) and methanol (500 μl) containing 100 μg of butylated hydroxytoluene (BHT). The mixture was vortexed for approximately 20 sec. Ethyl acetate (750 μl) was then added and the mixture was revortexed. The suspension was centrifuged at 3,000×g for 5 min. The organic (upper) layer was then transferred to a 1.5 ml microcentrifuge vial. Ethyl acetate (500 μl) was added to the residual aqueous phase and centrifuged as above. The organic layers were then pooled and concentrated by evaporation to a final volume of approximately 100 μl under a nitrogen stream. Hydroperoxides were quantitated by the FOX2 method¹⁷. Samples (100 μl) were mixed with 900 μl FOX2 reagent (100 μM xylenol orange, ammonium ferrous sulfate 250 μM, 25 mM H₂SO₄ in 90% v/v methanol) and incubated at room temperature for 30 min in a 1.5 ml microcentrifuge vials. After centrifugation at 12,000×g for 5 min to remove any flocculated material, absorbance of the supernatant was then read at 560 nm. FIG. 11B shows the amount of hydroperoxides in various brain tissues (cortex, striatum, hippocampus, erebellum) that were pre-treated with curcumin and then induced by T-BuOOH (T-BuOOH+curcumin), T-BuOOH alone without pre-treatment with curcumin (T-BuOOH), and negative control or normal rat.

Results

As shown in FIG. 11A, intracerebroventricular administration of the potent oxidant T-BuOOH in rats was progressively lethal in a high percentage (60%) of the treated animals over the course of 24 h. In contrast, pre-administration for 7 days of 50 mg/kg curcumin resulted in a marked protection, where only 20% of the pre- curcumin treated animals died. FIG. 11B shows the analysis of the lipid peroxides in different brain areas of treated animals, indicating that T-BuOOH induced significant alterations in the brain oxidative status. Pretreatment with curcumin prevented the oxidative damages to neuronal tissues.

### Example 4

Protective Effects of Cape in Models of Cerebral Neurodegeneration

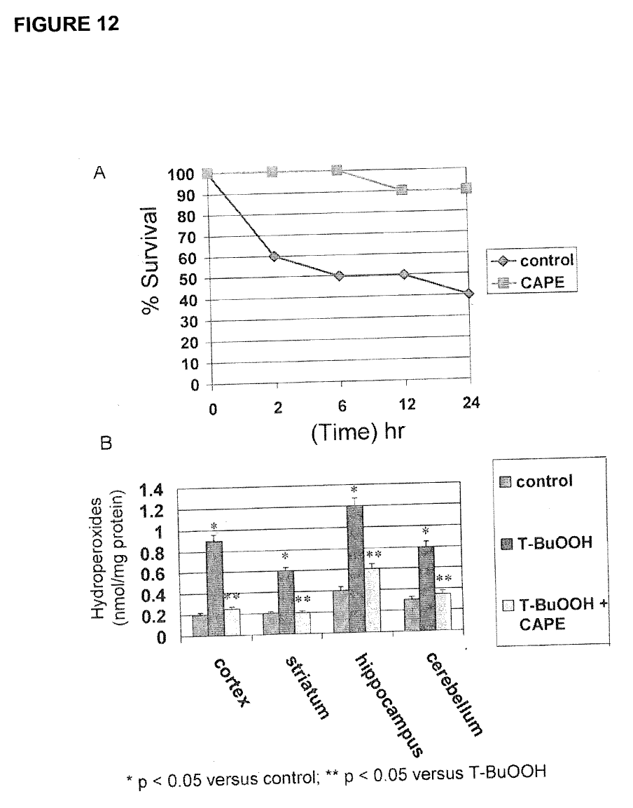

Male Wistar rats, weighing 140-180 g, were used for all experiments. A group of animals (n=10) was pre-administrated CAPE, suitably suspended in distilled water, at the dose of 20 mg/kg per day, through oral intubation, for 7 days, before an intracerebroventricular injection of T-butylhydroperoxide (T-BuOOH) 2 μl of a 70% solution of PBS as previously described. Lethality was assessed after administration of T-BuOOH at time points: 0, 2, 6, 12, and 24 h, and was expressed as the percentage of survival relative to the lethality observed in T-BuOOH plus Tween/saline vehicle (control) treated animals (FIG. 12A). After 30h the surviving animals were sacrificed and their brains quickly removed to investigate the lipid peroxides formation as an index of oxidative challenge in neurons (FIG. 12B).

Results

Pre-administration for 7 days of 20mg/kg CAPE resulted in a marked protection against T-BuOOH induced lethality (FIG. 12A). T-BuOOH induced significant alteration in the brain oxidative status, as demonstrated by the analysis of the lipid peroxides in different brain areas of treated animals (FIG. 12B). Pretreatment with CAPE prevented oxidative damages to neuronal tissues.

### Example 5

Neuroprotective and Anti-apoptotic Effects of Polyphenolic Compounds in Astrocytes

Chemicals and Reagents

Curcumin and CAPE were purchased from Sigma Chemical (St. Louis, Mo.). The chemical structures of these phenolic compounds are shown in FIG. 1. Curcumin-95, a commercially available mixture of curcuminoids (68% curcumin, 17% dimethoxy curcumin, 3% bis-dimethoxy curcumin, and 12% other curcumins), was purchased from Advanced Orthomoleuclar Research (Smith Falls, ON, Canada). Stock solutions of curcumin and other polyphenolic compounds were prepared as described previously¹¹. N-Acetyl-L- cysteine (NAC), reduced (GSH) and oxidized (GSSG) glutathione, and all other reagents were from Sigma unless otherwise specified. Rabbit polyclonal antibodies directed against HO-1 were obtained from Stressgen (Victoria, Canada).

Cell Culture

Type 1 astrocytes (DI TNC1) were purchased from the American Type Culture Collection (Manassas, VA) and cultured in Dulbecco's modified Eagle's medium containing 4.5 g/l glucose, 2 mM glutamine, 100 units/ ml penicillin, and 0.1 mg/ml streptomycin and supplemented with 10% fetal bovine serum. Cells were grown in 75-cm² flasks and maintained at 37° C. in a humidified atmosphere of air and 5% CO₂. Confluent cells were exposed to various concentrations of curcumin, CAPE, or Curcumin-95. After each treatment (6 or 24 h), cells were harvested for the determination of heme oxygenase activity, HO-1 protein expression, and intracellular glutathione. Astrocytes growing in 24 wells were exposed to polyphenolic compounds, and cell viability was determined at 24 h.

Heme Oxygenase Activity Assay

Heme oxygenase activity was determined at the end of each treatment as described previously by Foresti, et al.¹⁸ and Motterlini, et al.¹⁹ Briefly, microsomes from harvested cells were added to a reaction mixture containing NADPH, glucose-6-phosphate dehydrogenase, rat liver cytosol as a source of biliverdin reductase, and the substrate hemin. The reaction mixture was incubated in the dark at 37° C. for 1 h and was terminated by the addition of 1 ml of chloroform. After vigorous vortex and centrifugation, the extracted bilirubin in the chloroform layer was measured by the difference in absorbance between 464 and 530 nm (ε=40 mM⁻¹cm⁻¹).

Western Blot Analysis

After treatment with curcumin, or CAPE, samples of astrocytes were also analyzed for HO-1 protein expression using a Western immunoblot technique described previously^{18,19}. Briefly, an equal amount of proteins (30 μg) for each sample was separated by SDS-polyacrylamide gel electrophoresis and transferred overnight to nitrocellulose membranes, and the non-specific binding of antibodies was blocked with 3% non-fat dried milk in PBS. Membranes were then probed with a polyclonal rabbit anti-HO-1 antibody (Stressgen) (1:1000 dilution in Tris-buffered saline, pH 7.4) for 2 h at room temperature. After three washes with PBS, blots were visualized using an amplified alkaline phosphatase kit from Sigma (Extra-3A), and the relative density of bands was analyzed by the use of an imaging densitometer (model GS-700; Bio-Rad, Herts, UK). Blots shown are representative of three independent experiments.

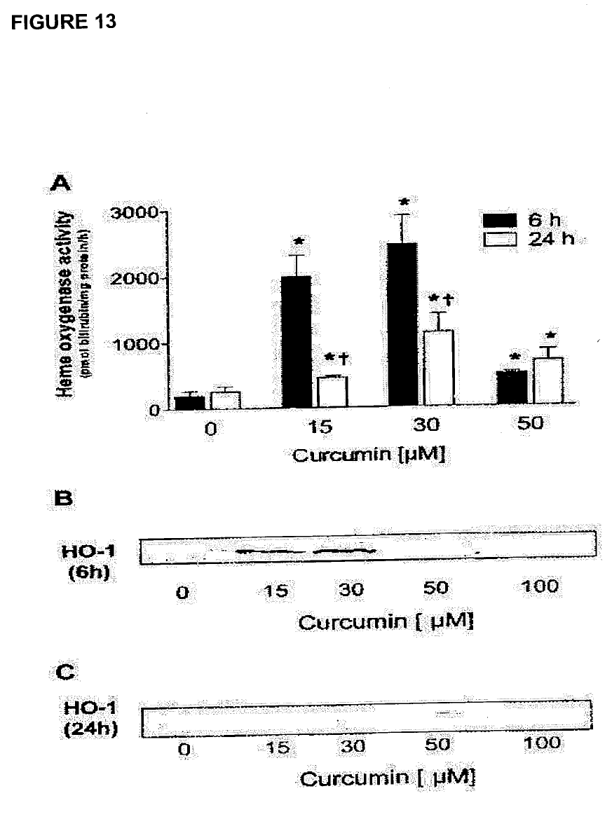

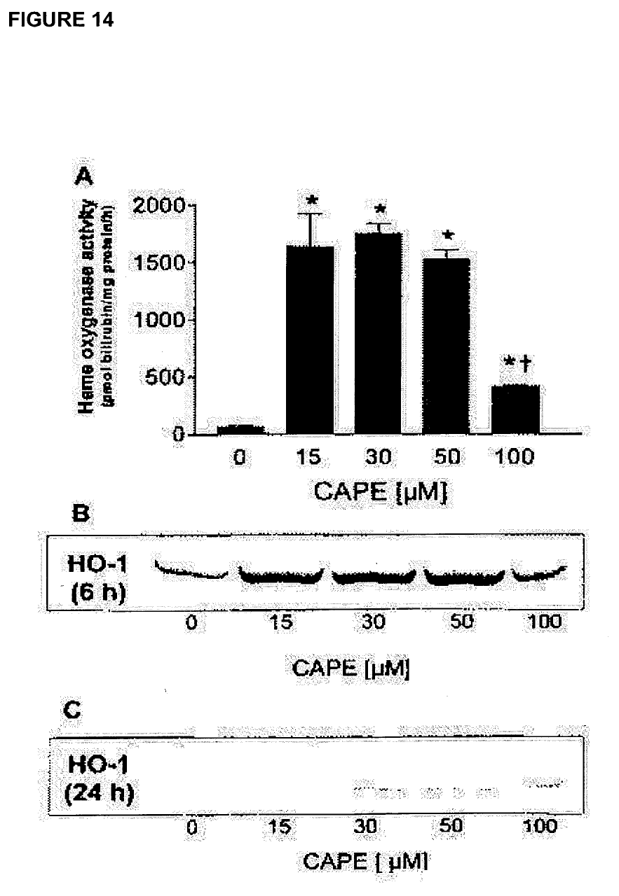

FIG. 13c shows that exposure of astrocytes for 6 h to 15 and 30 μM curcumin resulted in a gradual and significant (p<0.05) increase in heme oxygenase activity (7.4-and 9.1-fold, respectively). This enzymatic activation observed upon astrocyte exposure to curcumin was strongly associated with a marked up-regulation of HO-1 protein, as confirmed by Western blot analysis. Although to a lesser extent, over-expression of HO-1 was also found in astrocytes 24 h after curcumin treatment. In contrast, curcumin failed to increase HO-1 expression when higher concentrations (50-100 μM) of this drug were used. Consequently, the elevation in heme oxygenase activity was much less pronounced (1.9-fold). Similar to the effect evoked by curcumin, exposure of cells to low concentrations of CAPE (15-50 μM) resulted in a substantial increase in heme oxygenase activity and HO-1 protein levels (FIG. 14c). Maximal enzyme activation and protein expression were found at 30 μM CAPE, whereas 100 μM was significantly less effective. The reduced ability of curcumin and CAPE to increase heme oxygenase activity at high concentrations (50-100 μM) correlated with a cytotoxic effect exerted by these two drugs.

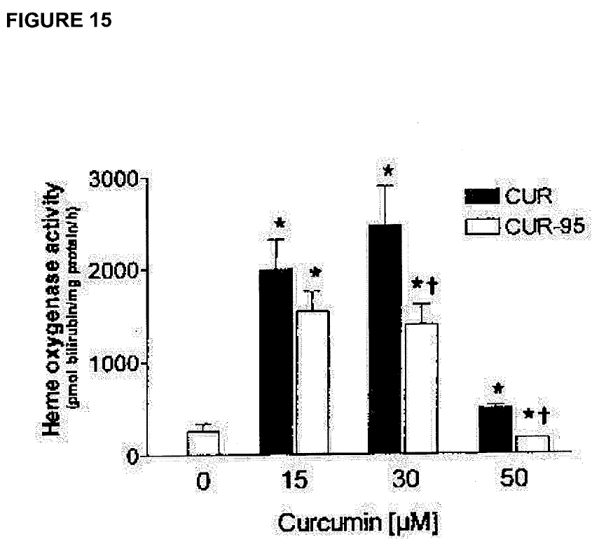

It is interesting that the exposure of astrocytes for 6 h to low concentrations of Curcumin-95 (15-30 μM), a mixture of curcuminoids that is commercially available as a dietary supplement, also resulted in a significant elevation of heme oxygenase activity compared with controls (FIG. 15c). However, this effect was less pronounced compared with pure curcumin. Similar to the effect caused by pure curcumin, high concentrations of Curcumin-95 (50 μM) did not cause any significant increase in heme oxygenase activity.

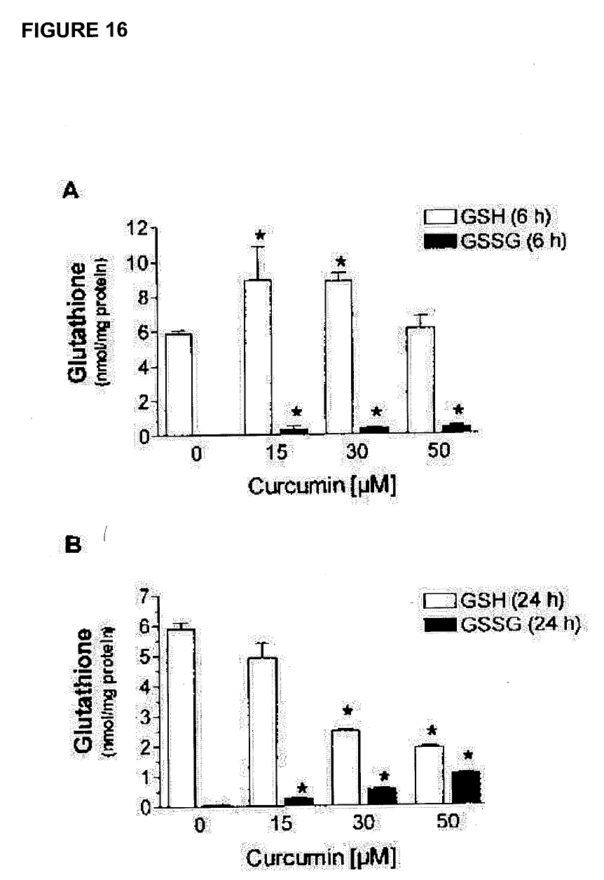

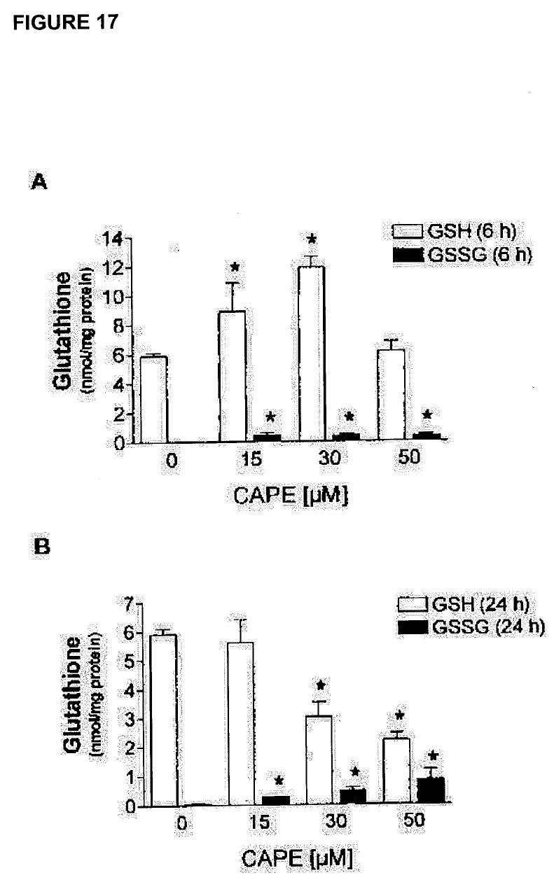

The potency of CAPE and curcumin in increasing HO-1 expression and consequently heme oxygenase activity upon addition to astrocytes may be associated with a rapid change in the intracellular redox status. Despite and initial oxidation of glutathione (GSSG) after exposure of astrocytes to low doses of curcumin and CAPE, this treatment did not significantly affect cell viability. Moreover, at concentrations that caused a gradual increase in heme oxygenase activity (15 and 30 μM), both CAPE and curcumin promoted an early increase in GSH levels, and this effect was reflected in the maintenance of cell viability even after prolonged incubations with the two agents. In the early stages of the treatment with high concentrations (50 and 100 μM) of curcumin and CAPE, a significant loss in cell viability was associated with a failure to increase the GSH content and was accompanied by a late and more dramatic reduction in the GSH/GSSG ratio (FIGS. 16c and 17c). At the high concentrations, CAPE and curcumin were unable to stimulate an increase in heme oxygenase activity. These results are consistent with the notion that transient and moderate changes in the redox status of the cell are prerequisites for the induction of cytoprotective genes (such as HO-1) and that a more severe oxidation inflicted to GSH results in suppression of the cellular stress response, ultimately leading to cell death²¹.

Effect of N-Acetyl-L-Cysteine on Curcumin-Mediated Activation of Heme Oxygenase

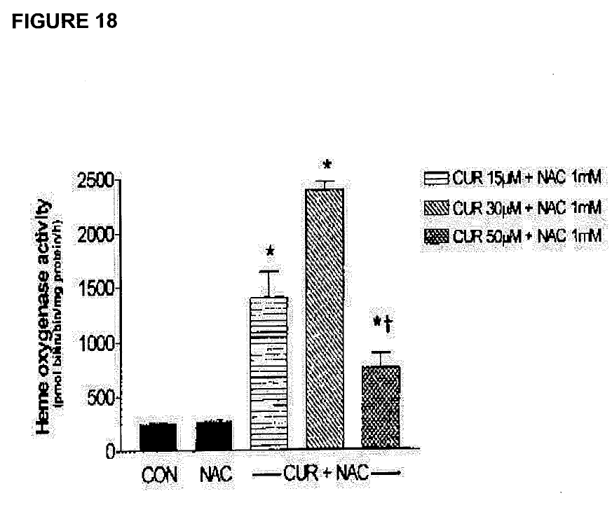

To determine the role of thiols in the modulation of heme oxygenase acitviyt by phenolic compounds, cells were exposed to various concentrations of curcumin for 6 h in the present of 1 mM N-acetyl-L-cysteine (NAC), a precursor of glutathione synthesis that possesses anti-oxidant properties. As shown in FIG. 18c, the substantial increase in heme oxygenase activity observed with both 15 and 30 μM curcumin was not significantly affected by the presence of NAC. At 30 μM, for instance, curcumin increased heme oxygenase activity from 247±5 (control) to 2461±194 pmol of bilirubin/mg of protein/h (p<0.05), and the addition of NAC to the culture medium did not change the potency of activation by this phenolic agent (2392±22 pmol of bilirubin/mg of protein/h). Similar results were obtained when astrocytes were incubated with CAPE in the presence of NAC. At higher concentrations of curcumin (50 μM), the increase in heme oxygenase activity was less pronounced at 492±30 and 752±78 pmol of bilirubin/mg of protein/h in the absence or presence of NAC, respectively. The fact that NAC, a precursor of glutathione synthesis with potent anti-oxidant properties, significantly attenuated the loss of cell viability but failed to prevent HO-1 express mediated by CAPE and curcumin indicate that HO-1 induction, in these circumstances, may no be directly related to redox changes involving glutathione.

Cell Viability Assay

Astrocytes were exposed to curcumin or CAPE for the indicated times, and cell viability was assessed with the use of an Alamar Blue assay according to manufacturer's instructions (Serotec; Oxford, UK) as reported previously¹¹. At the end of each treatment, cells were washed twice and incubated for an additional 5 h in complete medium containing 1% Alamar Blue solution. Optical density in each sample was measured using a plate reader (Molecular Devices; Crawley, UK). The intensity of the color developed in the medium is proportional to the viability of cells, which is calculated as the difference in absorbance between 570 and 600 nm and expressed as percentage of control.

Effect of Curcumin, CAPE and Curcumin-95 on Cell Viability

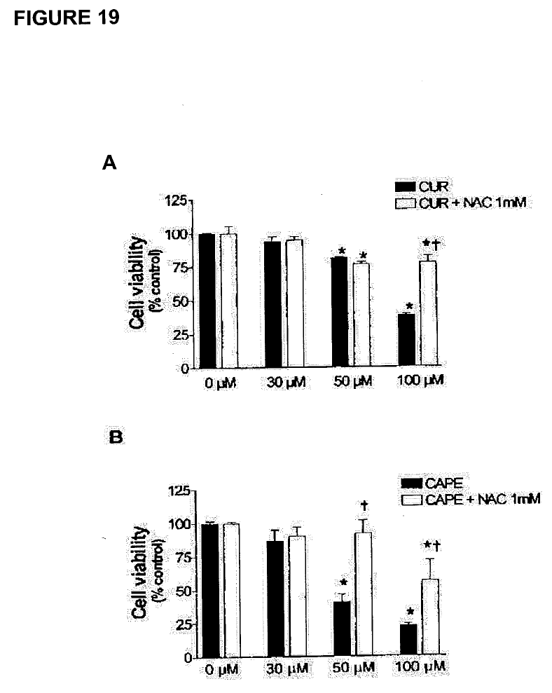

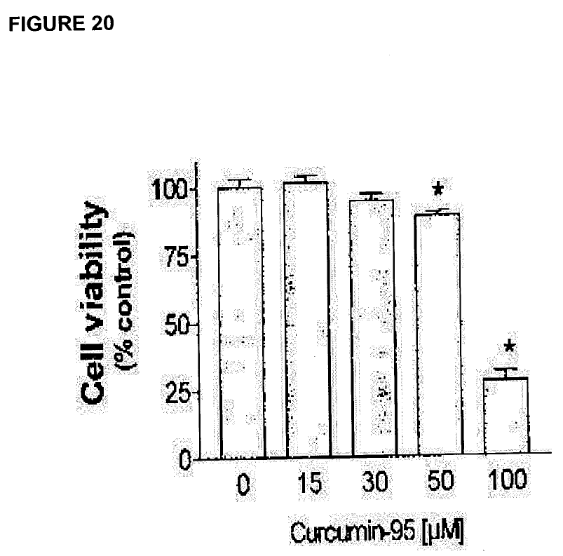

To determine a potential toxic effect of phenolic compounds on astrocytes, cell grown to confluence in 24 wells were incubated with increasing concentrations of curcumin, CAPE or Curcumin-95 for 24 h. When the concentration of these drugs did not exceed 30 μM, cell viability (determined using the Alamar Blue assay) as well as cell morphology observed under the microscope were fully preserved throughout the incubation period (FIGS. 19c and 20c). In contrast, treatment of astrocytes with 50 and 100 μM curcumin was cytotoxic, causing 20 and 63% reductions in cell viability, respectively (FIG. 19cA). A similar pattern was observed after exposure of astrocytes to 50 and 100 μM Curcumin-95, which promoted 21 and 69% losses in viability, respectively (FIG. 20c). The toxic effect of CAPE was more pronounced because treatment with this drug at 50 μM and 100 μM resulted, respectively, in 61 and 78% reductions in the number of viable cells. The presence of 1 mM NAC in the culture medium significantly attenuated the cytotoxic action mediated by both curcumin (100 μM) and CAPE (50 μM and 100 μM).

Determination of Intracellular Glutathione

GSH and GSSG levels were measured after 6- and 24- h exposure of astrocytes to curcumin and CAPE using a method previously described^{19,20}. Briefly, cells harvested in cold PBS were freeze-thawed three times, and an aliquot of this suspension was added to a buffer solution containing 12 mM EDTA and 10 mM 5,5′-dithiobis-(2-nitrobenzoic acid). Total glutathione was measured spectrophotometrically (optical density=412nm) using the glutathione reductase-recycling assay. To determine the amount of GSSG, an aliquot of the cell suspension was added to an equal volume of buffer containing EDTA and N-ethylmaleimide (10 mM). The sample was mixed and centrifuged, and the supernatant was passed through a C18 Sep-Pak cartridge (Waters, Milford, Mass.) to remove the excess N-ethylmaleimide. The sample was added to a cuvette containing 5,5′-dithiobis-(2-nitrobenzoic acid) and glutathione reductase, and the assay was performed as for the measurement of total glutathione. Intracellular glutathione was determined by comparison with a standard curve obtained with GSH and GSSG solutions and was expressed as nmoles/mg of protein.

Effect of CAPE and Curcumin on Intracellular Glutathione Levels

To determine the effect of polyphenolic compounds on the redox status of the cell, GSH and GSSG levels were determined at 6 and 24 h after treatment of astrocytes with different concentrations of curcumin and CAPE. Exposure to 15 and 30 μM curcumin for 6 h resulted in a significant increase in both intracellular GSH and GSSG, whereas 50 μM caused oxidation without affecting the GSH content (FIG. 8). A prolonged exposure (24 h) to curcumin (15, 30, and 50 μM) caused a concentration-dependent decrease in GSH that was paralleled by a gradual and substantial increase in GSSG levels. CAPE (15 and 30 μM) evoked a similar effect on intracellular glutathione leading ot the elevation of GSH in the early stage of the treatment followed by a marked reduction at 24 h (FIG. 9). Once again, exposure of cells to 50 μM CAPE did not affect GSH at 6 h, whereas prolonged incubation (24 h) caused a significant depletion of GSH and concomitant elevation in GSSG (p<0.05 versus control).

Statistical Analysis

Differences in the data among the groups were analyzed by using one-way analysis of variance combined with the Bonferroni test. Values were expressed as the mean ±S.E.M., and differences between groups were considered to be significant at p<0.05.

The above description of various preferred embodiments has been presented for purposes of illustration and description. It is not intended to be exhaustive or limiting to the precise forms disclosed. Obvious modifications or variations are possible in light of the above teachings. The embodiments discussed were chosen and described to provide illustrations and its practical application to thereby enable one of ordinary skill in the art to utilize the various embodiments and with various modifications as are suited to the particular use contemplated. All such modifications and variations are within the system as determined by the appended claims when interpreted in accordance with the breadth to which they are fairly, legally and equitably entitled.

### REFERENCES

1. Trauth B C, Klas C, Peters A M, Matzku S, Moller P, Falk W, Debatin K M, Krammer P H. (1989) Science 1989 Jul. 21; 245(4915):301-5.

2. Practico D, et al. (1998) FASEB J 12(15):1777-1783.

3. Ammon H P T and Wahl M A (1991) Planfa Med 57:1-7.

4. Huang M T, Smart R C, Wong C Q and Conney A H (1988) Cancer Res 48: 5941-5946.

5. Huang M T, Newmark H L and Frenkel K (1997) J Cell Biochem Suppl 27: 26-34.

6. Dinkova-Kostova A T and Talalay P (1999) Carcinogenesis 20: 911-914.

7. Natarajan K, Singh S, Burke T R, Jr, Grunberger D and Aggarwal B B (1996) Proc Nati Acad Sci USA 93: 9090-9095.

8. Michaluart P, Masferrer J L, Carothers A M, Subbaramaiah K, Zweifel B S, Koboldt C, Mestre J R, Grunberger D, Sacks P G, Tanabe T, et al. (1999) Cancer Res 59: 2347-2352.

9. Frenkel K, Wei H, Bhimani R, Ye J, Zadunaisky J A, Huang M T, Ferraro T, Convey A H and Grunberger D (1993) Cancer Res 53: 1255-1261.

10. Huang M T, Ma W, Yen P, Xie J G, Han J, Frenkel K, Grunberger D and Convey A H (1996) Carcinogenesis 17: 761-765.

11. Motterlini, et al. (2000) Free Radic Biol Med 28(8):1303-1312.

12. Scapagnini, et al. (2002) Mol. Pharmacol. 3(61):554-561.

13. Chen K, Gunter K 8 Maines MD (2000) J Neurochem 75:304-312.

14. Chang J, Rao N V, Markewitz B A, Hoidal J R, Michael J R (1996) Am J Physiol 270(6 Pt 1): L931-40.

15. Maines M D (1996) Meth Enzymol 268: 473-488.

16. Adams, J. D. et al.(1993) Free Radic Biol Med 15,195-202.

17. Calabrese V et al. (2002) J Neurosci Res 68, 65-67.

18. Foresti, et al. (1997) J. Biol. Chem. 272: 18411-18417.

19. Motterlini, et al. (2000) J. Biol. Chem. 275:13613-13620.

20. Calabrese, et al. (1998) Free Radic Biol Med 24:1159-1167.

21. Motterlini, et al. (2002) Antioxid Redox Signal 4:615-24.

What is claimed is:

1. A method of protecting neuronal cells, central and peripheral nervous system cells, and cells associated therefrom, from cell death in a subject, said method comprising administering an effective amount of a polyphenolic compound, or analog thereof, to said subject, sufficient to protect the neuronal cells, central and peripheral nervous system cells, and those associated thereof, from cell death.

2. The method according to claim 1, wherein the polyphenolic compound is selected from the group consisting of: curcumin, caffeic acid phenethyl ester, and analogs thereof.

3. The method according to claim 1, wherein the cell death is by apoptosis.

4. A method of inducing activity of a neuroprotective protein in a subject, said method comprising administering an effective amount of a polyphenolic compound, or analog thereof, to said subject, sufficient to induce activity of a neuroprotective protein.

5. The method according to claim 4, wherein the neuroprotective protein protects neuronal cells, central and peripheral nervous system cells, and cells associated therefrom, from oxidative damage.

6. The method according to claim 5, wherein the neuroprotective protein is selected from the group consisting of: heme oxygenase-1 and heat shock protein 70.

7. The method according to claim 4, wherein the polyphenolic compound is selected from the group consisting of: curcumin, caffeic acid phenethyl ester, and analogs thereof.

8. A method of preventing or treating a disease associated with cell death in neuronal cells, central and peripheral nervous system cells, and cells associated therefrom, in a subject, said method comprising administering an effective amount of a polyphenolic compound, or analog thereof, to said subject, sufficient to prevent or treat a disease associated with cell death in neuronal cells, central and peripheral nervous system cells, and cells associated therefrom.

9. The method according to claim 8, wherein the polyphenolic compound is selected from the group consisting of: curcumin, caffeic acid phenethyl ester, and analogs thereof.

10. The method according to claim 8, wherein the disease associated with cell death is a neurodegenerative disease.

11. The method according to claim 10, wherein the neurodegenerative disease is selected from the group consisting of: diseases, disorders, and conditions related to excessive activation of excitatory amino acid receptors or the generation of free radicals in the brain which cause nitrosative or oxidative stress, including aging, stroke, cerebral ischemia and hypoxia ischemia, hypoglycemia, domoic acid poisoning, anoxia, carbon monoxide or manganese or cyanide poisoning, central nervous system infections, meningitis, dementia, HIV-mediated dementia, Huntington's disease, Alzheimer's disease, Parkinson's disease, head and spinal cord trauma, epilepsy, seizures, and convulsions, olivopontocerebellar atrophy, demyelinating diseases, amyotrophic lateral sclerosis (ALS), meningitis, multiple sclerosis, neuropathic pain, diabetic neuropathy and HIV-related neuropathy, mitochondrial diseases, MERRF and MELAS syndromes, Leber's disease, Wernicke's encephalophathy, Rett syndrome, homocysteinuria, hyperprolinemia, hyperhomocysteinemia, nonketotic hyperglycinemia, hydroxybutyric aminoaciduria, sulfite oxidase deficiency, combined systems disease, and lead encephalopathy), Tourette's syndrome, hepatic encephalopathy, drug addiction, drug tolerance, drug dependency, depression, anxiety, and schizophrenia.


SEQUENCE LISTING (4 sequences)

SEQ ID NO: 1 - DNA, 19 bp, Artificial Sequence
  SYNTHETIC PRIMER
tgctcgcatgaacactctg

SEQ ID NO: 2 - DNA, 19 bp, Artificial Sequence
  SYNTHETIC PRIMER
tcctctgtcagcagtgcct

SEQ ID NO: 3 - DNA, 20 bp, Artificial Sequence
  SYNTHETIC PRIMER
caccactgcactttacttca

SEQ ID NO: 4 - DNA, 20 bp, Artificial Sequence
  SYNTHETIC PRIMER
agtgctggggagttttagtg